SEQUENCE LISTING (14 sequences)

SEQ ID NO: 1 - DNA, 1102 bp, Arabidopsis thaliana
atggcgagtttcatcaaggcatggggtttagtgatcatctcactgtgttacacttttttcattgccaaattggttccaaaaggaatcaaaaggctcatactatttttccctgtcttcctcattttcttcatagtacctttcttgatatattccttacatttactcggcatcacggctttcttcatcgcttggctagcaaatttcaagctcttattatttgcattagggcgcggtcctctctcttcaaaccataaacccctatctctccctattttcttagctgtctcttgcttgcccatcaagattcagctgagcccaaaacctacaaaaactcactcccatgaaggatccacagagggtcctttgatttataccataaaggcagtttttgtggttctcatcatcaaagcctacgaatacagtaccaaattgcctgagaaagtcgtgctgactctctacgcgatccacatatatttcgcccttgagatcatccttgccgccacagctgctgcggttcgagccatgtcggatcttgagctcgagccacagttcaacaagccgtacctagcgacatcacttcaagatttctgggggagacgatggaacctgatggtcactggaatcttacggccaaccgtgtacgaaccgtcacttcaactgttctcggttttgggcccgaactattcccagattcttgcagctttcgggacgtttgttgtctctgggataatgcacgagctcatcttcttctacatgggacggttgaggccagactggaagatgatgtggttcttcctcataaatggattttgcacgaccgtggagatcgccatcaagaaaaccattaacggtaggtggagattcccgaaagcaatcagtcaggttttgacactcacttttgtgatggtgacggcattgtggctgttcttgcccgaatttaatcggtgcaacatagttgagaaggctcttgatgagtacgcagccataggcgcatttgcagtcgaggtcaggaggaaactgaccgcatatcttttttaaccctgccgtgtgacaaagcttgagtgatctctattttaacttatttttttctaacactgtaaaa

SEQ ID NO: 2 - DNA, 1038 bp, Arabidopsis thaliana
atggcgagtttcatcaaggcatggggtttagtgatcatctcactgtgttacacttttttcattgccaaattggttccaaaaggaatcaaaaggctcatactatttttccctgtcttcctcattttcttcatagtacctttcttgatatattccttacatttactcggcatcacggctttcttcatcgcttggctagcaaatttcaagctcttattatttgcattagggcgcggtcctctctcttcaaaccataaacccctatctctccctattttcttagctgtctcttgcttgcccatcaagattcagctgagcccaaaacctacaaaaactcactcccatgaaggatccacagagggtcctttgatttataccataaaggcagtttttgtggttctcatcatcaaagcctacgaatacagtaccaaattgcctgagaaagtcgtgctgactctctacgcgatccacatatatttcgcccttgagatcatccttgccgccacagctgctgcggttcgagccatgtcggatcttgagctcgagccacagttcaacaagccgtacctagcgacatcacttcaagatttctgggggagacgatggaacctgatggtcactggaatcttacggccaaccgtgtacgaaccgtcacttcaactgttctcggttttgggcccgaactattcccagattcttgcagctttcgggacgtttgttgtctctgggataatgcacgagctcatcttcttctacatgggacggttgaggccagactggaagatgatgtggttcttcctcataaatggattttgcacgaccgtggagatcgccatcaagaaaaccattaacggtaggtggagattcccgaaagcaatcagtcaggttttgacactcacttttgtgatggtgacggcattgtggctgttcttgcccgaatttaatcggtgcaacatagttgagaaggctcttgatgagtacgcagccataggcgcatttgcagtcgaggtcaggaggaaactgaccgcatatcttttttaa

SEQ ID NO: 3 - protein, 345 aa, Arabidopsis thaliana
MASFIKAWGLVIISLCYTFFIAKLVPKGIKRLILFFPVFLIFFIVPFLIYSLHLLGITAFFIAWLANFKLLLFALGRGPLSSNHKPLSLPIFLAVSCLPIKIQLSPKPTKTHSHEGSTEGPLIYTIKAVFVVLIIKAYEYSTKLPEKVVLTLYAIHIYFALEIILAATAAAVRAMSDLELEPQFNKPYLATSLQDFWGRRWNLMVTGILRPTVYEPSLQLFSVLGPNYSQILAAFGTFVVSGIMHELIFFYMGRLRPDWKMMWFFLINGFCTTVEIAIKKTINGRWRFPKAISQVLTLTFVMVTALWLFLPEFNRCNIVEKALDEYAAIGAFAVEVRRKLTAYLF

SEQ ID NO: 4 - DNA, 820 bp, Brassica napus
ggcgctagctcgccgcagccgaacgaccgagcgcagcgagtcagtgagcgaggaagcggccgcataacttcgtatagcatacattatacgaagttatcagtcgacggtaccggacatatgcccgggaattcggccattacggccggggtggttcgagccatgtcaggtcttgagctagagccacagttcaacaaaccgtacctggcgacatctcttcaggatttctgggggagacgatggaacctggcggtcactggaatcctaaggcctaccgtgtacgagccgatgattcagctcttctctgtcttggacccgaactggtcacgtgttcttgctgttctcgccacgtttgttgtctcagggttgatgcacgagctcatcctcttctacatgggagggttgatgccggattggaaggtaatgtggttcttccttgtacacgggctctgcactactgtggagatcgccgtcaagaaaaaagtcaacgggaggtggaggcttccgacaggagtcggtcgtgtttgacgttcgggtttgtcatggtgacgggtttgtggctgttcttgccggtgtttaatcggggcaacatatttgagagggctcttgaagagtatgcatatgcaggcgcgcttgcagccgagctcaagaccatcatggcatctctactttaaatacttgcccatgaaaacaaatcttgagtttcatattgctacaaaacatatcaagaatcatgatcacatcgcatgcgactctttatttgtcttctgttgatacaacagttctaaagtttcttcaaaaaaaaaaaaaaaaaaaaaaaaaa

SEQ ID NO: 5 - protein, 174 aa, Brassica napus
GASSPQPNDRAQRVSERGSGRITSYSIHYTKLSVDGTGHMPGNSAITAGVVRAMSGLELEPQFNKPYLATSLQDFWGRRWNLAVTGILRPTVYEPMIQLFSVLDPNWSRVLAVLATFVVSGLMHELILFYMGGLMPDWKVMWFFLVHGLCTTVEIAVKKKVNGRWRLPTGVGRV

SEQ ID NO: 6 - protein, 130 aa, Arabidopsis thaliana
AATAAAVRAMSDLELEPQFNKPYLATSLQDFWGRRWNLMVTGILRPTVYEPSLQLFSVLGPNYSQILAAFGTFVVSGIMHELIFFYMGRLRPDWKMMWFFLINGFCTTVEIAIKKTINGRWRFPKAISQV

SEQ ID NO: 7 - protein, 130 aa, Brassica napus
AITAGVVRAMSGLELEPQFNKPYLATSLQDFWGRRWNLAVTGILRPTVYEPMIQLFSVLDPNWSRVLAVLATFVVSGLMHELILFYMGGLMPDWKVMWFFLVHGLCTTVEIAVKKKVNGRWRLPTGVGRV

SEQ ID NO: 8 - protein, 9 aa, Artificial
  Consensus sequence
DFWGRRWNL

SEQ ID NO: 9 - protein, 334 aa, Vitis vinifera
MKSFGYVWILAIASFCYCYFISASIPKGLFRLLSLLPIIFLFTTLPLHLSAFHPCGLTAFLLVWLANFKLLLFSFGRGPLSPPQPLLHFICTASLPIKISQNPHPNSYKITSPYSKTGQKVAFLIKALALAALLKVYKYRQFLHPNVILALYCCHVYLAAELILALAAAPARAIGLELEPQFNEPYLATSLQDFWGRRWNLMVSSILRPTIYFPIRGFTASGLGPRCSHLLAMLAAFTVSGLMHEVIYYYLTRVTPTWEVTWFFVLQGVCTAAEVAAKKAAAGRWRFPPAVTRPLTVVFVAVTSFWLFFPQLLRNHVDVKTIGEYSILIDFVKE

SEQ ID NO: 10 - protein, 334 aa, Vitis vinifera
MKSFGYVWILAIASFCYCYFISASIPKGLFRLLSLLPIIFLFTTLPLHLSAFHLCGLTAFLLVWLANFKLLLFSFGRGPLSPPQPLLHFICTASLPIKISQNPYPNSYKITSPYSKTGQKVAFLIKALALAALLKVYKYRQFLHPNVILALYCCHVYLAAELILALAAAPARAIGLELEPQFNEPYLATSLQDFWGRRWNLMVSSILRPTIYFPIRGFTASRLGPRCSHLLAMLAAFTVSGLMHEVIYYYLTRVTPTWEVTWFFVLQGVCTAAEVAAKKAAAGRWQLPPAVTRPLTVVFVAATGFWLFFPQLLRNHVDVKTIGEYSILLDFVKE

SEQ ID NO: 11 - protein, 321 aa, Vitis vinifera
IKVCLSVLASLCYSYFIVSKIPKGKFRLLSLLPIFSLFVALPLFLSTAILSGITAFFITWLATFRLALFSFDLGPLSTGSPKSLLVFIAIACLPIKIKPNQQHPSRQDPHKPPRLPLNFAVKVLAFGVFIGFYQYKELVHPKIFLGLLCCQVFLFLEVLFSLCSALVRCTTGLEVEQPSDEPYLSTLLQDFWGRRWNLMVTNLLRHTVYKPVKSAAETVMSERWSPLPAVVATFLVSGLMHELLFYYVNRVSPSWEMTSFFVLHGVCLVVEVGVKSVFSGRWRLHWAASVPLTVGFVVATSFWLFFPPLIRAGADMRVMEE

SEQ ID NO: 12 - protein, 262 aa, Oryza sativa
GCSAFFLSWLGVFKLLLLAAGRGPLNPTHPLHHFVFSASLPVKLRHLASAKPAKGVDPAPANESAAGKILVSGAVIPLIIYTYQFKNAMSRYQLLILYTGHIYFSLQLLLAVVHGLIHGVLGMEMEPQVDRPYLASSLRDFWGRRWNLMVPAILRPSVYRPVRARLGDAAGVLAAFLVSGLMHEAMFFYIMWRPPSGEVTVFFLLHGVCTAAEAWWARHAGWWRPPRAAAVPLTLAFVAGTGFWLFFPAMIKAGLDEMVLHE

SEQ ID NO: 13 - protein, 262 aa, Oryza sativa
GCSAFFLSWLGVFKLLLLAAGRGPLNPTHPLHHFVFSASLPVKLRHLASAKPAKGVDPAPANESAAGKILVSGAVIPLIIYTYQFKNAMSRYQLLILYTGHIYFSLQLLLAVVHGLIHGVLGMEMEPQVDRPYLASSLRNFWGRRWNLMVPAILRPSVYRPVRARLGDAAGVLAAFLVSGLMHEAMFFYIMWRPPSGEVTVFFLLHGVCTAAEAWWARHAGWWRPPRAAAVPLTLAFVAGTGFWLFFPAMIKAGLDEMVLHE

SEQ ID NO: 14 - protein, 321 aa, Oryza sativa
LRSLVAVCAAVTAAMWCARFAARRLRPGLPRLAAFVPVLAVLPFLPLAFRALHPRAISGFFLAWLAEFKLLLLASGQGPLDPSLPLPAFVAIATFPVRQRDPTKNAAGSGLGPVTSAVMAALLAAIVSLYRYKERMNPYALLVLYSLHVYLALELVLACAAAAVRAVMGMDLEPQFDRPYLSAHLRDFWGRRWNLSVPAVLRPCVSHPVRARVGEGAAGFAAGVLAAFFVSGVMHELMFYYITLRPPTGEATAFFTLHGALAVAEGWWAAREGWPRPPRPVATALTLALVMSTGFWLFFPPITRAGADKVVIAESEAVVAF

(19) United States
(12) Patent Application Publication
(10) Pub. No.: US 2010/0064381 A1
(43) Pub. Date: Mar. 11, 2010

(54) Increased Phytosterol Content Through Overexpression of an Acyl-CoA Sterol Acyl-Transferase

(76) Inventors: Jitao Zou, Saskatoon (CA); Qilin Chen, Saskatoon (CA)

Correspondence Address: NATIONAL RESEARCH COUNCIL OF CANADA;1200 MONTREAL ROAD, BLDG M-58, ROOM EG12, OTTAWA, ONTARIO, K1A 0R6, CA

(21) Appl. No.: 12/225,827

(22) PCT Filed: Dec. 8, 2006
(86) PCT No.: PCT/CA2006/002155

§ 371 Date: Jun. 2, 2009

(30) Foreign Application Priority Data
Mar. 30, 2006 (CA) PCT/CA2006/000476

(51) Int. Cl. A01H 1/00; A01H 5/10; C07J 53/00; C12N 5/10

(52) U.S. Cl. 800/261; 800/278; 800/298; 552/514; 435/419

(57) ABSTRACT

The present invention relates to the use of genetic engineering to produce sterol esters. In certain embodiments, an isolated or recombinant nucleic acid molecule encoding a sterol acyltransferase is disclosed. In certain other embodiments, a cell transformed with the isolated or recombinant nucleic acid molecule encoding a sterol acyltransferase is disclosed. A process for producing sterol esters using the transformed cell is also disclosed. In a further embodiment, an isolated or recombinant sterol acyltransferase is disclosed.

### CROSS-REFERENCE TO RELATED APPLICATIONS

This application is a continuation-in-part of PCT International Patent Application Serial No. PCT/CA2006/000476, filed Mar. 30, 2006, designating the United States of America, and published, in English, as PCT International Publication No. WO 2006/102755 A1 on Oct. 5, 2006, which application claims priority to U.S. Provisional Patent Application Ser. No. 60/666,520, filed Mar. 30, 2005, the contents of the entirety of each of which are hereby incorporated herein by this reference.

### TECHNICAL FIELD

The present invention relates generally to biotechnology and, more particularly, to the use of genetic engineering to overexpress an Acyl-CoA sterol acyltransferase to increase the production of phytosterol ester content in plants.

### BACKGROUND

The ability of phytosterols to lower low density lipoprotein (“LDL”) cholesterol in human subjects as part of a diet has been established in the medical field. Phytosterol-containing foods having therapeutic value have been approved for use by the Food and Drug Administration (“FDA”) and are available on the market.

However, free phytosterols are difficult to incorporate into food stuffs due to the low solubility of the free phytosterols. Phytosterol esters can, however, be dissolved in oil at a concentration ten times greater than that of the free phytosterols. Thus, the current commercial production of phytosterol-containing foods utilizes a costly fatty acid acylation procedure.

There have been various attempts to manipulate sterols in plants. For instance, PCT International Patent Publication WO 98/45457 (the contents of which are incorporated by this reference) describes modulating phytosterol compositions to confer resistance to parasites and/or environmental stresses, and/or to improve the nutritional value of plants by using a double stranded DNA molecule comprising a promoter, a DNA sequence encoding a first enzyme which binds a first sterol and produces a second sterol and a 3′ non-translated region which causes polyadenylation at the 3′ end of the RNA.

U.S. Pat. No. 5,306,862 (the contents of which are incorporated by this reference) describes a method of increasing sterol accumulation in a plant by increasing the copy number of a gene encoding a polypeptide having HMG-CoA reductase activity to increase the resistance of plants to pests.

U.S. Pat. No. 5,349,126 (the contents of which are incorporated by this reference) discloses a process to increase the squalene and sterol accumulation in transgenic plants by increasing the amount of a gene encoding a polypeptide having HMG-CoA reductase activity to increase the pest resistance of transgenic plants. WO 96/09393 (the contents of which are incorporated by this reference) discloses a DNA sequence encoding squalene synthetase. WO 97/34003 (the contents of which are incorporated by this reference) discloses a process of raising squalene levels in plants by introduction into a genome of a plant a DNA to suppress expression of squalene epoxidase.

PCT International Patent Publication WO 97/48793 (the contents of which are incorporated by this reference) discloses a C-14 sterol reductase polypeptide for the genetic manipulation of a plant sterol biosynthetic pathway.

PCT International Patent Publication WO 93/16187 (the contents of which are incorporated by this reference) discloses plants containing in their genomes one or more genes involved in the early stages of phytosterol biosynthesis, preferably the genes encode mevalonate kinase.

U.S. Pat. No. 5,589,619 (the contents of which are incorporated by this reference) discloses accumulation of squalene in plants by introducing a HMG-CoA reductase gene to increase production of sterol and resistance to pests.

PCT International Patent Publication WO 00/08190 (the contents of which are incorporated by this reference) discloses a DNA sequence encoding a sterol methyltransferase isolated from Zea mays.

U.S. Patent Publication 20040172680 (the contents of which are incorporated by this reference) describes the use of a gene expressing a SMT1 to increase the level of sterols in the seeds of plants and/or decrease the level of cholesterol in plant tissue.

Further, since phytosterol esterification processes in planta may represent one biochemical bottleneck that limits phytosterol biosynthesis and, hence, the amount of phytosterols produced, a need exists for a more efficient process for the production of sterol esters.

### SUMMARY OF THE INVENTION

In certain embodiments described herein, a sterol acyltransferase gene is identified. The sterol acyltransferase gene may be under-expressed, expressed or overexpressed in a cell and used to enhance sterol-ester production in the cell. In certain other embodiments, the sterol acyltransferase gene is expressed or overexpressed in planta in order to enhance the production of sterol esters in a crop. In an additional embodiment, a plant, plant seed or progeny thereof, includes the sterol acyltransferase gene.

In certain other embodiments, the sterol acyltransferase gene may be expressed or overexpressed in a cell and used to enhance the biosynthesis and accumulation of sterols in the cell. In an additional embodiment, the sterol acyltransferase gene is expressed or overexpressed in planta in order to increase the total content of sterols in a crop. In certain further embodiments, a plant, plant seed or progeny thereof, includes the sterol acyltransferase gene.

In certain other embodiments, a vector having a sterol acyltransferase gene is disclosed. The vector may be used to transform a cell, thus producing a recombinant cell having the sterol acyltransferase gene. The cell may comprise, for example, a bacterial cell, a yeast cell or a plant cell. In certain other embodiments, the cell expresses the sterol acyltransferase gene and produced a sterol acyltransferase peptide that may be isolated or purified from the cell. The isolated or purified sterol acyltransferase peptide may be used to generate antibodies having utility in diagnostics or further studies.

In certain further embodiments, the nucleotide and deduced amino acid sequence associated with a sterol acyltransferase gene are disclosed. The sequence, or a portion of which, may be used to identify genes from other species that encode a polypeptide with sterol acyltransferase activity. The nucleotide sequence may be used to transform a cell, thus producing a recombinant cell having the sterol acyltransferase gene. The cell may comprise a bacterial cell, a yeast cell or a plant cell.

In certain embodiments, a process for producing sterol esters includes transforming a cell with a sterol acyltransferase gene. The transformed cell expresses the sterol acyltransferase gene and produces sterol esters. The sterol esters may be isolated or purified from the recombinant cell or culture media in which the cell grows and subsequently incorporated into a composition as described herein.

In yet an additional embodiment, the sterol esters produced with the process of the instant invention are administered in combination with the active ingredients of pharmaceutical or nutraceutical compositions such as, for example, cholesterol-lowering agents. Non-limiting examples of cholesterol-lowering agents include, without limitation, plant sterols, psyllium, beta glucan, niacin, guggul extract, red rice yeast extract, statin(s), policosanol, garlic, fenugreek, rice bran oil, fish oil, flaxseed oil, borage oil, other omega-3-fatty acid-containing oils, and combinations of any thereof.

In an additional exemplary embodiment, the sterol esters produced by the processes of the instant invention are incorporated in a food product, such as a beverage or a food, a multi-ingredient nutritional supplement or any combination thereof. Non-limiting examples of food products that the sterol esters may be incorporated into include cholesterol-lowering margarine, soy protein, nuts, flaxseed, olive oil, fish oil, any other oil, and combinations of any thereof.

The sterol esters produced by the process of the instant invention can be used directly as food additives or admixed with a consumable carrier to be used as the food additive or food composition. One food additive of the invention includes the sterol esters and a consumable carrier.

In certain other embodiments, an article of manufacture includes a container for holding an amount of a composition comprising sterol esters of the instant invention, and indicia associated with the container. The indicia are organized to guide a reader of the indicia to ingest an effective amount of the composition sufficient to help lower or reduce the cholesterol content of a subject that ingests the composition.

It is also contemplated that sterol esters produced by the process of the instant invention may be prepared in, for example, capsule, tablet or liquid form for regular administration to help treat conditions involving high cholesterol.

In certain other embodiments, the sterol esters produced by the process of the instant invention are administered as a pharmaceutical or nutraceutical composition to a subject. Such a composition includes the sterol esters and a pharmaceutically acceptable carrier such as, for example, lactose, cellulose, vitamin E, oils, or equivalent, or contained within a pharmaceutical dosage such as a capsule or tablet and may be used in combination with other pharmaceutical or nutraceutical active ingredients, or cosmetic ingredients that improve the appearance (e.g., color) of the sterol esters.

In certain embodiments, provided is an isolated or recombinant nucleic acid molecule encoding a plant sterol acyltransferase having at least 70% (more preferably, 80%, 90% or 95%) homology to SEQ ID NO:2 of the herein incorporated SEQUENCE LISTING.

In certain embodiments, provided is a cell transformed with the isolated or recombinant nucleic acid molecule as described above.

In certain embodiments, provided is a process for increasing sterol ester production in a cell, the process comprising: transforming a cell with a nucleic acid molecule encoding a sterol acyltransferase; and growing the cell under conditions wherein the sterol acyltransferase is expressed.

In certain embodiments, provided is an isolated plant sterol acyltransferase, comprising an amino acid sequence that is at least 70% (more preferably, 80%, 90% or 95%) homologous to SEQ ID NO:3.

In certain embodiments, provided is a plant having a genomic knock-out of a sterol acyltransferase gene.

In certain embodiments, provided is a method of identifying sterol acyltransferase genes comprising: (a)transforming a yeast mutant deficient in sterol ester synthesis with a nucleic acid molecule comprising a plant cDNA suspected of encoding a sterol acyltransferase; and (b) detecting sterol ester formation in the yeast, wherein presence of sterol esters indicates that the plant cDNA encodes a functional sterol acyltransferase.

In certain embodiments, the invention thus includes a process of increasing the level of cycloartenol in the seed of plants beyond that (e.g., by at least 10%, 20%, or 30% by weight) of wild-type seed, the process comprising: transgenically overexpressing an acyl-CoA sterol acyltransferase in the plants producing the seeds. Cycloartenol is a known phytosterol believed to have healthful attributes such as being useful as an antioxidant, to support prostate health, to treat menopause, and to lower cholesterol levels in subjects ingesting cycloartenol.

In certain embodiments, the invention thus includes a process of obtaining seeds, the process comprising: (a) transforming a plant by: i. transforming a plant cell with a recombinant DNA construct comprising a nucleic acid segment encoding acyl-CoA sterol acyltransferase and a promoter for driving the expression of the nucleic acid segment in the plant cell to form a transformed plant cell, ii. regenerating the transformed plant cell into a transgenic plant, and iii. selecting transgenic plants that have enhanced levels of cycloartenol in the seeds compared wild type strains of the same plant; (b) cultivating the transformed plant for one or more generations; and (c) harvesting seeds from plants cultivated per (b).

In certain embodiments, the invention thus includes a seed having enhanced levels of cycloartenol and produced by a plant having increased acyl-CoA sterol acyltransferase activity. Such a seed preferably has a total level of sterol esters of at least 0.400% of dry weight.

In certain embodiments, the invention thus includes a process for obtaining oil comprising enhanced levels of cycloartenol, the process comprising: extracting oil from seed of the invention.

In certain embodiments, the invention thus includes oil produced by a process of the invention, as well as a food product comprising the oil.

In certain embodiments, the invention thus includes a process to increase the level of sterols in the seeds of plants and/or decrease the level of cholesterol in plant tissue by increasing the expression of acyl-CoA sterol acyltransferase in the plants.

In certain embodiments, the invention thus includes a plant tissue having increased levels of cycloartenol, the plant tissue being derived from a plant having increased acyl-CoA sterol acyltransferase activity.

In certain embodiments, the invention thus includes a process for modulating phytosterol synthesis in a plant, the process comprising modulating the expression of acyl-CoA sterol acyltransferase in the plant so as to modulate phytosterol synthesis therein. In certain aspects, the invention thus further includes the use of, for example, AtSAT1 to modulate or enhance phytosterol biosynthesis in plants.

In certain embodiments, the process may be modified to further include incorporating, for expression in the plant, another nucleic acid sequence that enhances phytosterol content, for example, a nucleic acid sequence encoding a peptide having HMG-CoA reductase activity, a nucleic acid sequence encoding SMT1, a nucleic acid sequence encoding a peptide having mevalonate kinase activity, a nucleic acid sequence encoding a peptide that enhances early stages of phytosterol biosynthesis, a nucleic acid sequence encoding a peptide having sterol methyltransferase activity, a nucleic acid sequence encoding a peptide having squalene synthetase activity, a DNA to suppress expression of squalene epoxidase, a nucleic acid sequence encoding a C-14 sterol reductase peptide for the genetic manipulation of the plant sterol biosynthetic pathway, or any combination thereof.

In certain embodiments, the invention thus includes a seed of the type having phytosterols including cycloartenol therein, the improvement comprising having cycloartenol as the most prominent phytosterol in the seed.

### BRIEF DESCRIPTION OF THE DRAWINGS

FIG. 1 is a genetic map of pYES2.1/V5-His-TOPO®.

FIG. 2 is the nucleotide sequence of At3 g51970 (SEQ ID NO:1).

FIG. 3 is the nucleotide sequence of the CDS of the plant sterol acyltransferase gene of Arabidopsis (SEQ ID NO:2).

FIG. 4 is the predicted amino acid sequence of At3g51970 (SEQ ID NO:3).

FIG. 5 illustrates HPLC chromatograms demonstrating the production of sterol esters in cells transformed with At3g51970.

FIG. 6 is a typical gas chromatography profile of sterol saponified from sterol ester extracted from wild-type (“WT”) and AtSAT1 over-expression (“OE”) plant seeds.

FIG. 7 is a table depicting the seed phytosterol profile of the transgenic lines described herein overexpressing AtSAT1.

FIG. 8 is a bar graph depicting a comparison of representative SE-sterol species composition of WT and AtSAT1 over-expression plant lines (T1).

FIG. 9 is CDNA (partial) sequence of the B. napus sterol acyltransferase (SEQ ID NO: 4).

FIG. 10 is an amino acid (partial) sequence of the B. napus sterol acyltransferase (SEQ ID NO: 5).

FIG. 11 is amino acid sequence alignment between the B. napus (SEQ ID NO: 6), Arabidopsis sterol acyltransferases (SEQ ID NO: 7), and consensus sequence (SEQ ID NO:8).

### DETAILED DESCRIPTION OF THE INVENTION

All publications, patents and patent applications mentioned herein are specifically incorporated herein by this reference.

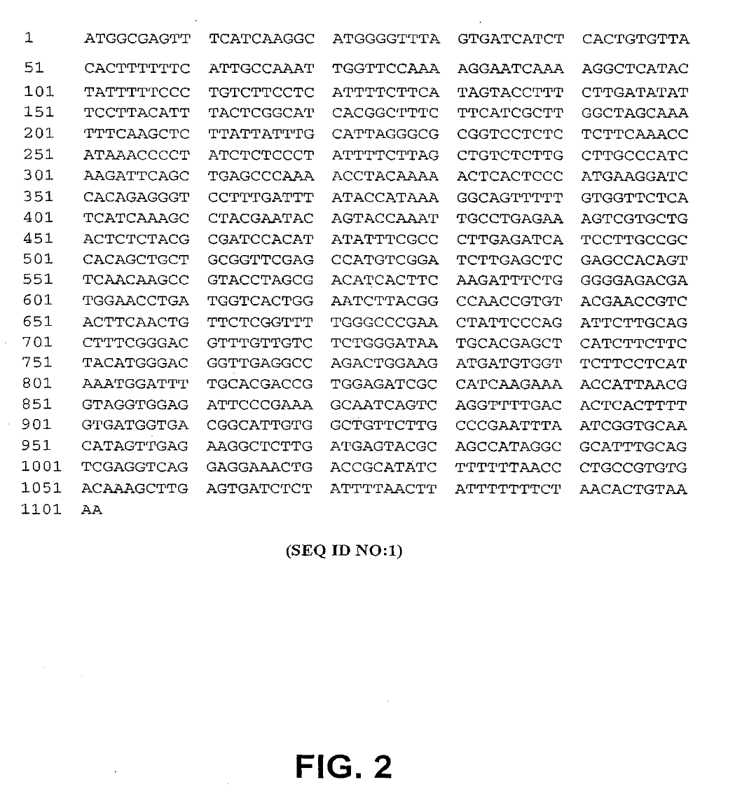

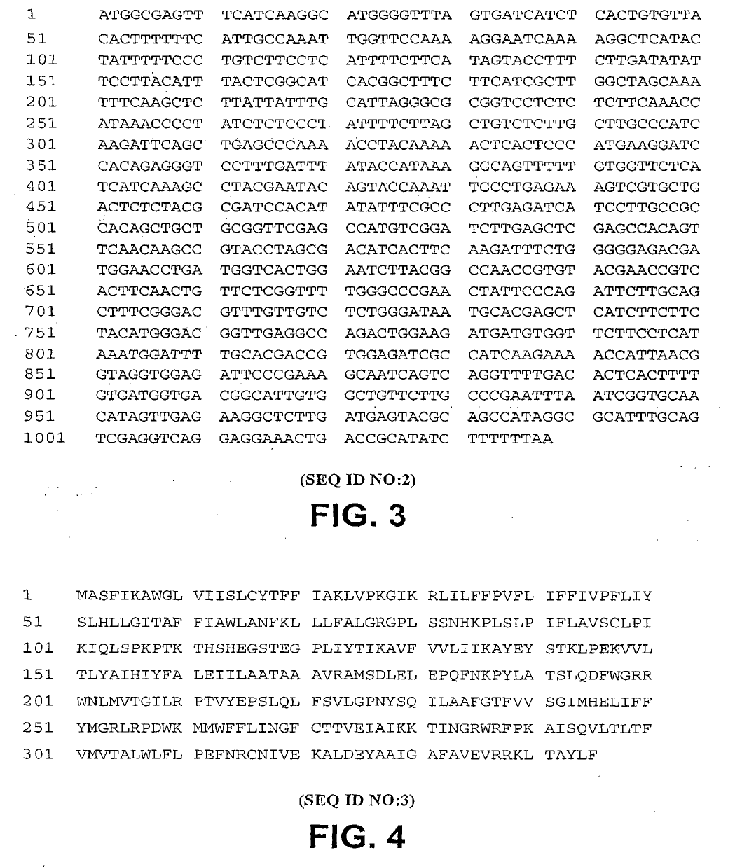

In certain embodiments, a sterol acyltransferase gene is identified with a yeast complementation approach in combination with high-performance liquid chromatography (“HPLC”) analysis of neutral lipids in a yeast extract. Specifically, the genomic sequence of the sterol acyltransferase gene is shown in FIG. 2 (SEQ ID NO:1), the coding sequence of the acyltransferase gene is shown in FIG. 3 (SEQ ID NO:2), and the amino acid sequence of the acyltransferase enzyme is shown in FIG. 4 (SEQ ID NO:3).

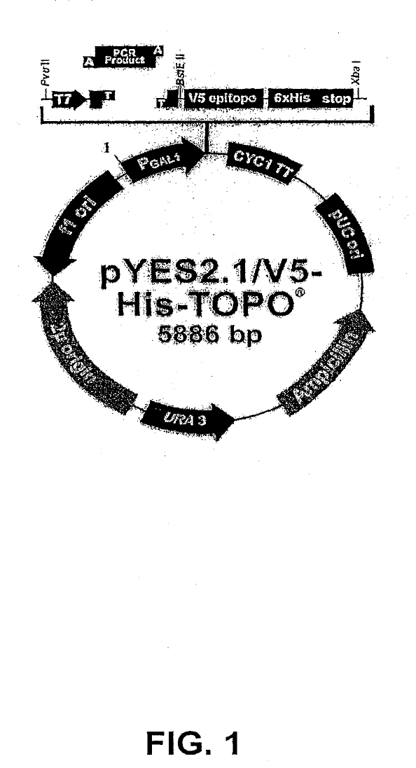

A class of membrane-binding 0-acyltransferase motif-containing genes may be found in Arabidopsis based on interrogation of a genomic database, combined with an assumption that phytosterol acyltransferase is a membrane-bound acyltransferase. Since not all of the cDNAs are expected to encode a functional sterol acyltransferase, a biochemical functional characterization of the gene products is performed in a yeast strain defective in sterol-ester production to discover the sterol acyltransferase gene. The members of cDNAs of the genes found with the interrogation are obtained by RT-PCR using seedling and/or silique RNA as a template. The cDNAs are cloned into a yeast-expression vector such as, for example, the readily and commercially available plasmid pYES2.1/V5-His-TOPO® (FIG. 1), and the plasmids having the cDNAs are introduced into a yeast mutant strain such as, for example, are1are2 that is deficient in sterol ester synthesis.

For selection, the transformed yeast is cultured in dropout SC medium that is -his-leu-ura at 28° C. for two days. The yeast-expression vector carries a URA gene :and the double mutant yeast itself is able to synthesize histidine and leucine. The yeast transformants, upon being induced by galactose, are subjected to normal-phase HPLC analysis of neutral lipid extracts to detect the distinct peak that corresponds to the sterol ester. If a peak appears at the very retention time of sterol ester, the gene whose products possess a function of acylating sterol is identified to produce sterol esters. As will be appreciated by one of skill in the art, other suitable mutants or combinations of mutants that are defective or deficient in sterol acylation may be used instead of are1are2.

In certain embodiments of the invention, provided is a method of identifying sterol acyltransferase genes comprising transforming a yeast mutant deficient in sterol-ester synthesis with a nucleic acid molecule comprising a plant cDNA suspected of, encoding a sterol acyltransferase; and detecting sterol-ester formation in the yeast, wherein presence of increased sterol-ester levels compared to a control indicates that the plant cDNA encodes a functional sterol acyltransferase.

As will be appreciated by one of skill in the art, the control may be an untransformed, mock-transformed or vector-transformed control and the control does not necessarily need to be repeated each time.

In those embodiments wherein the mutant is defective in sterol acylation, for example, are1are2, it is noted that simply the presence of sterol esters indicates that the plant cDNA encodes a functional sterol acyltransferase and no control is necessary.

In certain other embodiments, nucleotides that are at least 50%, 60%, 70%, 80%, 81%, 82%, 83%, 84%, 85%, 86%, 87%, 88%, 89%, 90%, 91%, 92%, 93%, 94% or 95% identical over their entire length to the polynucleotide encoding sterol acyltransferase of the instant invention, that is, to SEQ ID NO:1 or SEQ ID NO:2, and polynucleotides that are complementary to such polynucleotides, are disclosed.

In other embodiments, polynucleotides that encode polypeptides having substantially the same function as the sterol acyltransferase disclosed herein are disclosed. Conservative substitutions are specifically included.

In yet other embodiments, purified or isolated plant sterol acyltransferases comprising an amino acid sequence that is at least 60%, 65%, 70%, 75%, 76%, 77%, 78%, 79%, 80%, 81%, 82%, 83%, 84%, 85%, 86%, 87%, 88%, 89%, 90% or 95% homologous to SEQ ID NO:3 are disclosed. As will be appreciated by one of skill in the art, “isolated” refers to polypeptides that have been “isolated” from their native environment, in this case, from a plant cell, while “purified” does not necessarily refer to absolute purity but rather refers to at least a two-, three-, four- or five-fold increase in purity. It is further of note that methods for identification of such plant sterol acyltransferase are described herein.

In certain further embodiments, polynucleotides that hybridize to the above-disclosed sequences are disclosed. The hybridization conditions may be stringent in that hybridization will occur if there is at least a 90%, 95% or 97% identity with the polynucleotide that encodes the sterol acyltransferase of the instant invention. The stringent conditions may include those used for known Southern hybridizations such as, for example, incubation overnight at 42° C. in a solution having 50% formamide, 5×SSC (150 mM NaCl, 15 mM trisodium citrate), 50 mM sodium phosphate (pH 7.6), 5× Denhardt's solution, 10% dextran sulfate, and 20 micrograms/milliliter denatured, sheared salmon sperm DNA, followed by washing the hybridization support in 0.1×SSC at about 65° C. Other known hybridization conditions are well known and are described in Sambrook et al., Molecular Cloning: A Laboratory Manual, Third Edition, Cold Spring Harbor, N.Y. (2001), incorporated herein in its entirety by this reference.

In certain other embodiments, the present invention is directed towards homologs of the sterol acyltransferase gene described herein obtained from other organisms such as, for example, plants. Such homologs of the sterol acyltransferase gene described herein may be obtained by screening appropriate libraries that include the homologs, wherein the screening is performed with the nucleotide sequence of the plant sterol acyltransferase gene of the instant invention or portions or probes thereof.

In yet an additional embodiment, the nucleotide sequence of the sterol acyltransferase gene (FIG. 2, SEQ ID NO:1), the coding region (FIG. 3, SEQ ID NO:2), or the predicted amino acid sequence (FIG. 4, SEQ ID NO:3) of the instant invention may be used to search for homologous sequences using computer programs designed to search for homologous sequences. For instance, readily available commercial computer programs that may be used for such searches include without limitation, BLASTN, BLASTX and TBLASTX, which may be used to search for nucleotide sequences, and BLASTP and TBLASTN, which may be used to search for amino acid sequences. Such computer programs are readily accessible at the website www.ncbi.nlm.nih.gov.

Using a BLAST search described herein, several cDNAs from the public database were identified that we may be sterol acyltransferase from other plant species. See, for example, the Vitis vinifera wax synthase isoform 2 (WS-2) gene (accession AY174866 of the NCBI Entrez web data base (SEQ ID NO: 9)), V. vinifera wax synthase isoform 1 (WS-1) gene (accession AY174865 of the Entrez data base (SEQ ID NO: 10)), V. vinifera wax synthase isoform 3 (WS-3) gene (accession AY174867 of the Entrez data base (SEQ ID NO: 11)), Oryza sativa (japonica cultivar-group) Os02g0454500 (accession NM_{—}001053307 of the Entrez data base (SEQ ID NO: 12)) (see, “The map-based sequence of the rice genome” Nature 436 (7052), 793-800 (2005)), Oryza sativa (japonica cultivar-group) cDNA clone:002-103-F04 (accession AK064156 of the NCBI Entrez web data base (SEQ ID NO: 13)), and Oryza sativa (japonica cultivar-group) Os04g0481900 (accession NM_{—}001059650 of the Entrez data base (SEQ ID NO: 14)).

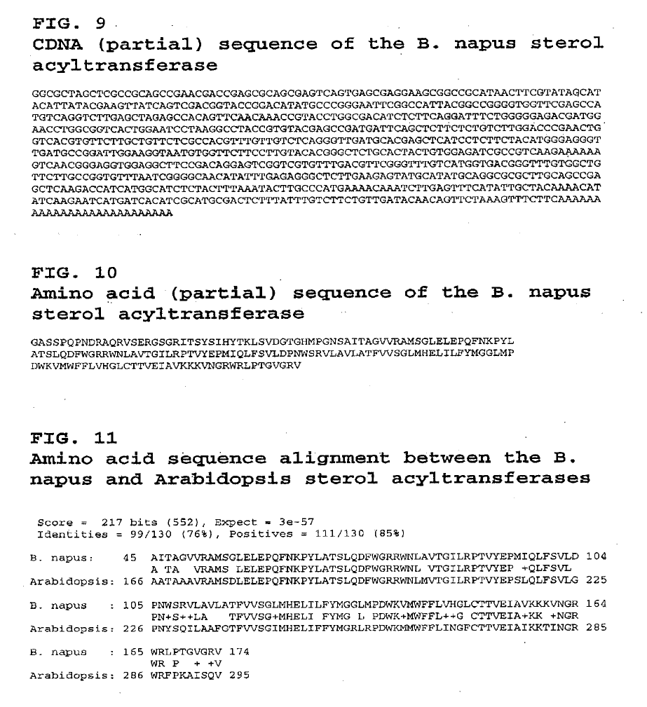

FIGS. 9 through 11 depict a partial cDNA encoding a canola (B. napus) sterol acyltransferase. Specifically, FIG. 9 is CDNA (partial) sequence of the B. napus sterol acyltransferase (SEQ ID NO: 4). FIG. 10 is an amino acid (partial) sequence of the B. napus sterol acyltransferase (SEQ ID NO: 5). FIG. 11 is amino acid sequence alignment between the B. napus (SEQ ID NO: 6) and Arabidopsis sterol acyltransferase-(SEQ ID NO: 7) including the consensus sequence (SEQ ID NO: 8).

In certain further embodiments, a nucleotide sequence that codes for a sterol acyltransferase is transformed into a plant. As known in the art, there are a number of ways by which genes and gene constructs can be introduced into plants, and a combination of plant transformation and tissue culture techniques have been successfully integrated into effective strategies for creating transgenic crop plants. These methods, which can be used in the present invention, have been described elsewhere (Fotrykus, 1991; Vasil, 1994; Walden and Wingender, 1995; Songstad et al., 1995), and are well known to persons skilled in the art. For example, one skilled in the art will certainly be aware that, in addition to Agrobacterium-mediated transformation of Arabidopsis by vacuum infiltration (Bechtold et al., 1993) or wound inoculation (Katavic et al., 1994), it is equally possible to transform other plant and crop species, using Agrobacterium Ti-plasmid-mediated transformation (e.g., hypocotyl (DeBlock et al., 1989) or cotyledonary petiole (Moloney et al., 1989) wound infection), particle bombardmentlbiolistic methods (Sanford et al., 1987; Nehra. et al., 1994; Becker et al., 1994) or polyethylene glycol-assisted protoplast transformation (Rhodes et al., 1988; Shimamoto et al., 1989) methods.

As will also be apparent to persons skilled in the art and as described elsewhere (Meyer, 1995; Dada et al., 1997), it is possible to utilize plant promoters to direct any intended up- or down-regulation of transgene expression using constitutive promoters (e.g., those based on CaMV35S), or by using promoters that can target gene expression to particular cells, tissues (e.g., napin promoter for expression of transgenes in developing seed cotyledons), or organs (e.g., roots) to a particular developmental stage or in response to a particular external stimulus (e.g., heat shock). The gene sequences of interest will be operably linked (that is, positioned to ensure the functioning of) to one or more suitable promoters which allow the DNA to be transcribed. Suitable promoters, which may be homologous or heterologous to the gene, useful for expression in plants are well known in art, as described, for example, in Weising et al., (1988), Ann. Rev. Genetics, 22, 421-477).

Promoters for use according to the invention may be inducible, constitutive or tissue-specific or have various combinations of such characteristics. Useful promoters include, but are not limited to constitutive promoters such as carnation etched ring virus (CERV), cauliflower mosaic virus (CaMV) 35S promoter, or more particularly the double enhanced cauliflower mosaic virus promoter, comprising two CaMV 35S promoters in tandem (referred to as a “Double 35S”promoter).

It may be desirable to use a tissue-specific or developmentally regulated promoter instead of a constitutive promoter in certain circumstances. A tissue-specific promoter allows for overexpression in certain tissues without affecting expression in other tissues. By way of illustration, a preferred promoter used in overexpression of enzymes in seed tissue is an ACP promoter as described in the hereby incorporated by this reference WO92/18634.

The promoter and termination regulatory regions will be functional in the host plant cell and may be heterologous (that is, not naturally occurring) or homologous (derived from the plant host species) to the plant cell and the gene. Suitable promoters which may be used are described above.

The termination regulatory region may be derived from the 3′ region of the gene from which the promoter was obtained or from another gene. Suitable termination regions which may be used are well known in the art and include Agrobacterium tumefaciens nopaline synthase terminator (Tnos), Agrobacterium tumefaciens mannopine synthase terminator (Tmas) and the CaMV 35S terminator (T35S). Particularly preferred termination regions for use according to the invention include the pea ribulose bisphosphate carboxylase small subunit termination region (TrbcS) or the Tnos termination region.

Such gene constructs may suitably be screened for activity by transformation into a host plant via Agrobacterium and screening for increased isoprenoid levels.

Suitably, the nucleotide sequences for the genes may be extracted from the Genbank nucleotide database and searched for restriction enzymes that do not cut. These restriction sites may be added to the genes by conventional methods such as incorporating these sites in PCR primers or by sub-cloning.

Preferably, the DNA construct according to the invention is comprised within a vector, most suitably an expression vector adapted for expression in an appropriate host (plant) cell. It will be appreciated that any vector which is capable of producing a plant comprising the introduced DNA sequence will be sufficient.

Suitable vectors are well known to those skilled in the art and are described in general technical references such as Pouwels et al., Cloning Vectors. A laboratory manual, Elsevier, Amsterdam (1986). Particularly suitable vectors include the Ti plasmid vectors.

Transformation techniques for introducing the DNA constructs according to the invention into host cells are well known in the art and include such methods as micro-injection, using polyethylene glycol, electroporation, or high velocity ballistic penetration. A preferred method for use according to the present invention relies on Agrobacterium-mediated transformation.

After transformation of the plant cells or plant, those plant cells or plants into which the desired DNA has been incorporated may be selected by such methods as antibiotic resistance, herbicide resistance, tolerance to amino-acid analogues or using phenotypic markers.

Various assays may be used to determine whether the plant cell shows an increase in gene expression, for example, Northern blotting or quantitative reverse transcriptase PCR (RT-PCR). Whole transgenic plants may be regenerated from the transformed cell by conventional methods. Such transgenic plants having improved isoprenoid levels may be propagated and self-pollinated to produce homozygous lines. Such plants produce seeds containing the genes for the introduced trait and can be grown to produce plants that will produce the selected phenotype.

Plants that may be modified or used for sterol ester production according to the instant invention include, without limitation, borage (Borago spp.), Canola, castor (Ricinus communis); cocoa bean (Theobroma cacao), corn (Zea mays), cotton (Gossypium spp.), Crambe spp., Cuphea spp., flax (Linum spp.), Lesquerella and Limnanthes spp., Linola, nasturtium (Tropaeolum spp.), Oeanothera spp., olive (Olea spp.), palm (Elaeis spp.), peanut (Arachis spp.), rapeseed, safflower (Carthamus spp.), soybean (Glycine and Sofa spp.), sunflower (Helianthus spp.), tobacco (Nicotiana spp.), Vernonia spp. wheat (Triticum spp.), barley (Hordeum spp.), rice (Oryza spp.), oat (Avena spp.) sorghum (Sorghum spp.), rye (Secale spp.) or other members of the Gramineae. Grape vines and rice may also be modified. It will further be apparent by those of ordinary skill in the art that genomic or sequence libraries of each of these plants may be screened with the nucleotide or amino acid sequences of the instant invention for other sequences that encode or are homologous to sequences associated with the plant sterol acyltransferase of the instant invention.

As will be appreciated by one of skill in the art, the level of sterol esters in plants are typically about 0.2% of total oil in seeds (or 0.5% free sterol plus sterol esters). As such, a transgenic plant comprising a non-native sterol acyltransferase gene as discussed herein will typically produce seeds having above 0.5% sterol esters.

In an additional embodiment, knock-out mutants of plants are constructed. The plants may be constructed by knocking out the nucleotide sequence in the genome of the plants encoding a sterol acyltransferase that is homologous to the sterol acyltransferase gene of the instant invention using known techniques.

In certain other embodiments, plants transformed with a nucleotide sequence of the instant invention that codes for a sterol acyltransferase and the knock-out mutants are studied for the impact of expressing the transformed nucleotide sequence that codes for the sterol acyltransferase or the lack of expression of the nucleotide sequence that codes for the sterol acyltransferase in knock-out mutants.

In certain further embodiments, plants transformed with a nucleotide sequence of the instant invention that codes for a sterol acyltransferase are grown. Seeds of the transgenic plants are harvested and sterol esters of the seeds are extracted. The extracted sterol esters are used for subsequent incorporation into a pharmaceutical composition, a nutraceutical composition or a food composition.

As previously identified, in certain embodiments, the process is modified to further include incorporating, for expression in the plant, another nucleic acid sequence that enhances phytosterol content. Such nucleic acids are known to those of skill in the art, for example, a nucleic acid sequence encoding a peptide having HMG-CoA reductase activity (see, e.g., the incorporated herein U.S. Pat. Nos. 5,306,862, 5,349,126, and 5,589,619), a nucleic acid sequence encoding SMT1 (see, e.g., the incorporated herein U.S. Patent Publication 20040172680), a nucleic acid sequence encoding a peptide that enhances early stages of phytosterol biosynthesis such as mevalonate kinase (see, e.g., the incorporated herein PCT International Patent Publication WO 93/16187), a nucleic acid sequence encoding a peptide having sterol methyltransferase activity (see, e.g., the incorporated herein PCT International Patent Publication WO 00/08190), a nucleic acid sequence encoding a peptide having squalene synthetase activity (see, e.g., the incorporated herein PCT International Patent Publication WO 96/09393), a DNA to suppress expression of squalene epoxidase (see, e.g., the incorporated herein PCT International Patent Publication WO 97/34003), a nucleic acid sequence encoding a C-14 sterol reductase peptide for the genetic manipulation of the plant sterol biosynthetic pathway (see, e.g., the incorporated herein PCT International Patent Publication WO 97/48793), or any combination thereof.

The invention will now be further explained and illustrated by way of the following illustrative Examples.

### Examples

### Example I

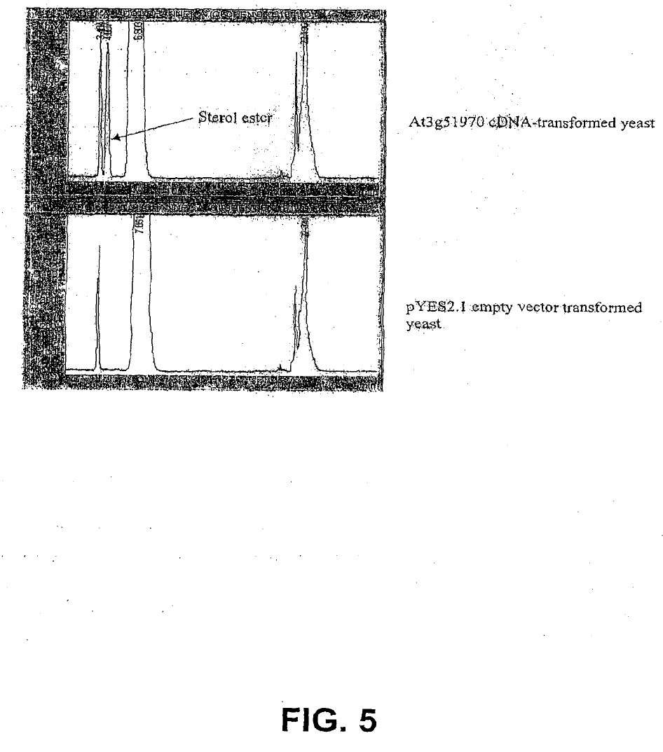

The Arabidopsis cDNA corresponding to the gene encoding a sterol acyltransferase, i.e., At3g51970 (FIG. 2), was cloned by RT-PCR using the commercial kit of “SuperScript First-Strand Synthesis System for RT-PCR,” commercially available from Invitrogen. The cDNA was sequenced and inserted into the vector pYES2.1/V5-His-TOPO® (FIG. 1), commercially available from Invitrogen and used according to the manufacturer's protocol. The vector having the inserted At3g51970 cDNA was transformed into the yeast are1/are2 mutant using the established procedure of Small-Scale Yeast Transformation as described in the pYES2.1/V5-His-TOPO® manual from Invitrogen. Neutral lipid extracts of the yeast were subjected to normal-phase HPLC analysis to assay for the production of sterol esters in the yeast. The top panel of FIG. 5 illustrates the production of sterol esters in the yeast transformed with At3g51970, while the vector lacking At3g51970, i.e., pYES2.1, served as a negative control that lacked the sterol esters as illustrated in the bottom panel of FIG. 5.

The Arabidopsis gene, At3g51970, is a novel plant sterol acyltransferase gene and the full-length coding sequence is shown in FIG. 3. The amino acid sequence of At3g51970 is shown in FIG. 4.

### Example II

### Identification of a Brassica sterol Acyltransferase Gene

The nucleotide and deduced amino acid sequence information from Arabidopsis is used to search against Brassica genomic, cDNA and/or Expressed Sequence Tag information to identify a sterol acyltransferase gene from other Brassica species, i.e., Brassica napus. In certain other embodiments, the gene and/or the cDNA of At3g51970 is used as a labeled probe to carry out nucleotide hybridization to identify genes encoding sterol acyltransferase. In yet another embodiment, the polypeptide that is produced or generated according to sequence information of At3g51970 is used to generate antibody that is used to screen a cDNA library for a sterol acyltransferase cDNA.

### Example III

### Transformation of a Plant with Plant Sterol Acyltransferase Gene

The transformation protocol is adapted from that described by Bechtold et al. (1993). Plants of Arabidopsis thaliana ecotype Columbia are grown in moist soil at a density of ten to twelve plants per pot, in four-inch square pots, and are covered with a nylon screen fixed in place with an elastic band. When the plants reach the stage at which bolts emerge, plants are watered, the bolts and some of the leaves are clipped, and the plants are infiltrated in Agrobacterium suspension as outlined below.

Agrobacterium transformed with the sterol acyltransferase gene of the instant invention is grown in a 25 mL suspension in LB medium containing kanamycin at a concentration of 50 μg/mL. The Agrobacterium is cultured for two to three days. The day before infiltration, this “seed culture” is added to 400 mL of LB medium containing 50 μg/mL kanamycin. When the absorbance at 600 nm is >2.0, the cells are harvested by centrifugation (5,000 times g, ten minutes in a GSA rotor at room temperature) and are re-suspended in three volumes of infiltration medium (1/2× Murashige and Skoog salts, 1× B5 vitamins, 5.0% sucrose, 0.044 μM benzylaminopurine) to an optical density at 600 nm of 0.8. The Agrobacterium suspension is poured into a beaker and the potted plants are inverted into the beaker so that the bolts, and entire rosettes are submerged. The beaker is placed into a large Bell jar and a vacuum is drawn using a vacuum pump until bubbles form on the leaf and stem surfaces and the solution starts to bubble a bit, and then the vacuum is rapidly released. The necessary time and pressure varies from one lab setup to the next, but good infiltration is visibly apparent as uniformly darkened, water-soaked tissue. Pots are removed from the beaker, laid on their side in a plastic tray and covered with a plastic dome to maintain humidity. The following day, the plants are uncovered, set upright and are allowed to grow for approximately four weeks in a growth chamber under continuous light conditions as described by Katavic et al. (1995). When the siliques are mature and dry, seeds are harvested and selected for positive transformants.

### Example IV

### Selection of Putative Transformants (Transgenic plants) and Growth and Analysis of Transgenic Plants

Seeds are harvested from vacuum-infiltration transformation procedures and are sterilized by treating for one minute in ethanol and five minutes in 50% bleach/0.05% Tween 20™ in sterile distilled water. The seeds are rinsed several times with sterile distilled water. Seeds are plated by re-suspending them in sterile 0.1% agarose at room temperature (about 1 mL agarose for every 500 to 1000 seeds) and applying a volume equivalent to about 2,000 to 4,000 seeds onto 150×15 mm selection plates (½× Murashige and Skoog salts, 0.8% agar, autoclave, cool and add 1×B5 vitamins and kanamycin at a final concentration of 50 μg/mL). The plates are dried in a laminar flow hood until seed no longer flows when the plates are tipped. The plates are vernalized for two nights at 4° C. in the dark and are moved to a growth chamber (conditions as described by Katavic et al., 1995). After seven to ten days, transformants are clearly identifiable as dark green plants with healthy green secondary leaves and roots that extend over and into the selective medium.

Seedlings are transplanted to soil, plants are grown to maturity and mature seeds (T2 generation as defined in Katavic et al., 1994) are collected and analyzed. T₂ seeds are propagated. The vegetative growth patterns are monitored by measuring shoot tissue dry weights and/or by counting the number of rosette leaves present by the time plants began to enter the generative (flower initiation) stage. Floral initiation (beginning of generative phase of growth) is analyzed by recording, on a daily basis, the percentage of plants in which a flower bud first appears and/or the percentage of plants that are bolting (as described by Zhang et al., 1997). Data is reported in terms of percentage of plants flowering/bolting on a given day after planting (d.a.p.).

### Example V

### Analysis of Sterol Esters

Cells or plants transformed with the sterol acyltransferase gene of the instant invention are grown to maturity and mature seeds are harvested. Neutral lipids are extracted from the cells or plants transformed with the sterol acyltransferase gene. The neutral lipids are subjected to normal-phase HPLC analysis to assay for the production of sterol esters in the transformed cells or plants.

### Example VI

### Comparison of WT and AtSAT1 OE Plants and Seeds

AtSAT1 was expressed in Arabidopsis in a seed-specific manner under the control a napin promoter, through which a large number of transgenic plants were generated. The phytosterol profile and content of 13 transgenic lines and several wild-type plants growing under identical conditions were then analyzed.

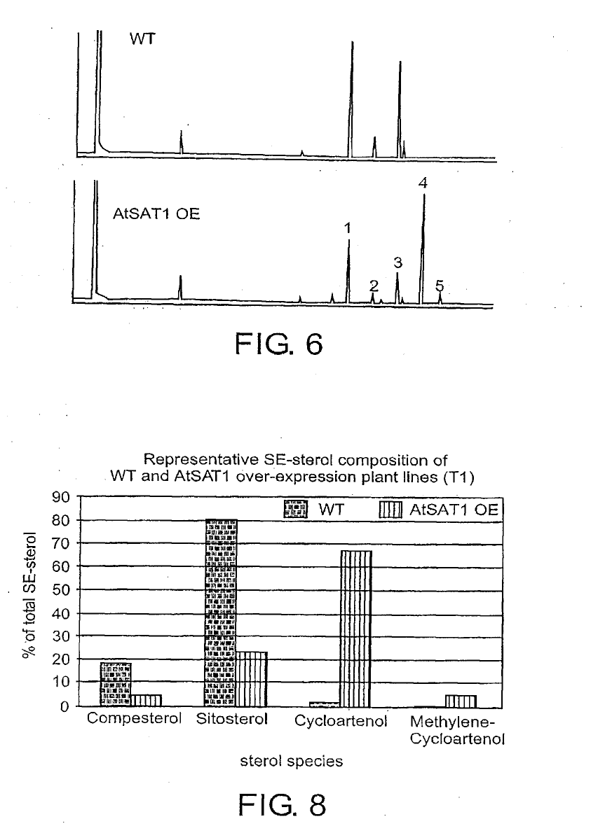

FIG. 6 is a typical gas chromatography profile of sterol saponified from sterol ester extracted from wild-type and AtSAT1 over-expression (“OE”) plant seeds. Neutral lipid species were extracted with chloroform:methanol (2:1) and separated on Baker Si250 TLC plate-silica gel. Bands containing sterol ester were scraped off and saponified with 7.5% KOH in 95% methanol. Freed sterol was derivatized by addition of bis(trimethylsilyl)trifluoro-acetamide (BSTFA):pyridine (1:1). The peaks in the profiles were subsequently identified by searching NIST 2.0 mass spectra library: 1, cholesterol (IS), 2. compesterol, 3. sitosterol, 4. cyclosterol, and 5. methylene cycloartenol.

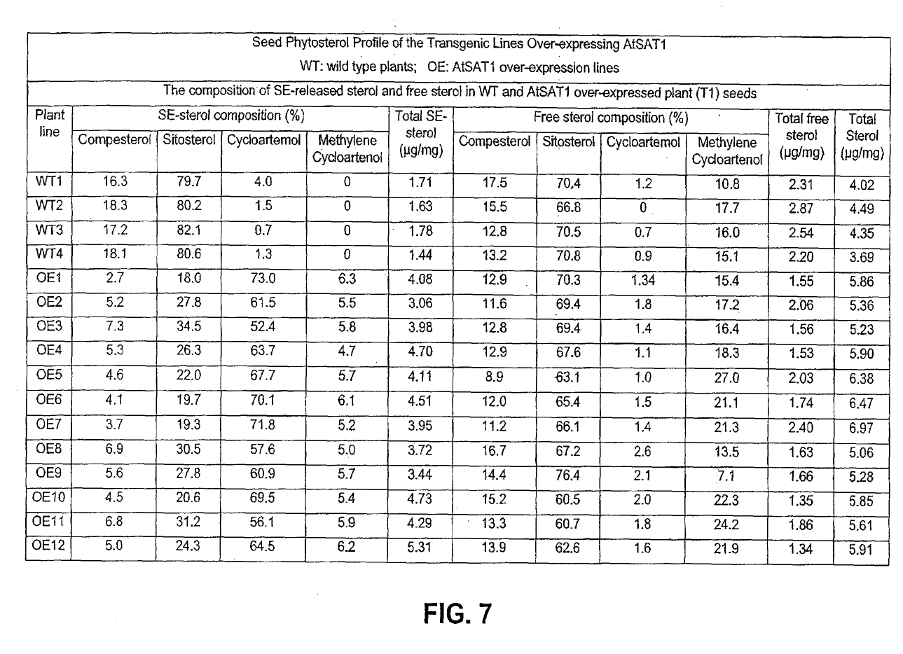

As can be seen, the results show that the overexpression of the AtSAT1 gene in seed resulted in dramatic and consistent changes in phytosterol biosynthesis in seeds. The results are tabularly presented in FIG. 7. FIG. 8 graphically depicts a comparison of representative SE-sterol species composition of WT and AtSAT1 over-expression plant lines. (T1).

Modifications of phytosterol synthesis was evidenced by both the profile and content of phytosterols found in the seeds. Cycloartenol, which is a minor component in wild-type seed, became the most prominent sterol species in the transgenic plants. Furthermore, while there was a smaller effect on free sterol content in the OE seeds. There was a two-fold increase in phytosterol ester content in some of the transgenic lines. The transgenic liens had as much as 0.45% (% seed weight) of sterol ester in seeds while the wild-type seeds have only 0.17%. The total sterol content (free sterol and sterol esters) in the transgenic lines reached 0.6%, which represented a 50% increase in comparison to that of wild-type seed (0.4%).

### Example VII

### Preparation and Analysis of Resulting Oil

OE seeds from Example VI are dried. The seeds are cleaned (e.g., by screening and washing) to remove stones, sand, dirt, and spoiled seeds. Any husk or seed coat is removed and seeds are separated from chaff. Optionally, the seed can be ground. Further optionally, the seed may be heated to increase efficiency of extraction and protein availability. Oil may be extracted oil mechanically with an oil press, expeller, or a wooden mortar and pestle. There are many different types of extractors. The extracted oil is allowed to stand for a few days and then upper layer is removed, clarifying the oil of contaminants. Further clarification may be done using, for example, a filter cloth or by boiling the oil. The oil is analyzed as per Example VI, and the oil is determined to have a greater concentration of cycloartenol than naturally occurring oils. The oil may be used to prepare a composition such as a food product, pharmaceutical and/or nutraceutical composition.

While the preferred embodiments of the invention have been described above, it will be recognized and understood that various modifications may be made therein, and the appended claims are intended to cover all such modifications that may fall within the spirit and scope of the invention.

### REFERENCES

### The Contents of the Entirety of Each of which are Incorporated by this Reference

Bechtold N., J. Ellis G. and Pellefer (1993). In planta Agrobacterium-mediated gene transfer by infiltration of adult Arabidopsis thaliana plants. C.R. Acad. Sci. Ser. III Sci. Vie, 316:1194-1199.
Becker D., R. Brettschneider and H. Lorz (1994). Fertile transgenic wheat from microprojectile bombardment of scutellar tissue. Plant J. 5:299-307.
Dada R., J. W. Anderson and G. Selvaraj (1997). Plant promoters for transgene expression. Biotechnology Annual Review 3:269-296.
DeBlock M., D. DeBrouwer and P. Terming (1989). Transformation of Brassica napus and Brassica oleracea using Agrobacterium tumefaciens and the expression of the bar and neo genes in the transgenie plants. Plant Physiol. 91:694-701.
Katavic Y., G. W. Haughn, D. Reed, M. Martin and L. Kunst (1994). In planta transformation of Arabidopsis thaliana. Mol. Gen. Genet. 245:363-370.
Meyer P. (1995). Understanding and controlling transgene expression. Trends in Biotechnology 13:332-337.
Moloney M. M., J. M. Walker and K. K. Sharma (1989). High efficiency transformation of Brassica napus using Agrobacterium vectors. Plant Cell Rep. 8:238-242.
Nehra N. S., R. N. Chibbar, N. Leung, K. Caswell, C. Mallard, L. Steinhauer, M. Baga and K. K. Kartha (1994). Self-fertile transgenic wheat plants regenerated from isolated scutellar tissues following microprojectile bombardment with two distinct gene constructs. Plant J. 5:285-297.
Potrykus L. (1991). Gene transfer to plants: Assessment of publish approaches and results. Annu. Rev. Plant Physiol. Plant Mol. Biol. 42:205-225.
Rhodes C. A., D. A. Pierce, I. J. Mettler, D. Mascarenhas and J. J. Detmer (1988). Genetically transfoiiued maize plants from protoplasts. Science 240:204-207.
Sanford J. C., T. M. Klein, E. D. Wolf and N. Allen (1987). Delivery of substances into cells and tissues using a particle bombardment process. J. Part. Sci. Technol. 5:27-37.
Shimamoto K., R. Terada, T. Izawa and H. Fujimoto (1989). Fertile transgenic rice plants regenerated from transformed protoplasts. Nature 335:274-276.
Vasil I. K. (1994). Molecular improvement of cereals. Plant Mol. Biol. 5:925-937.
Walden R. and R. Wingender (1995). Gene-transfer and plant regeneration techniques. Trends in Biotechnology 13:324-331.
Songstad D. D., D. A. Somers and R. J. Griesbach (1995). Advances in alternative DNA delivery techniques. Plant Cell, Tissue and Organ Culture 40:1-15.
Zhan H., H. M. Goodman and S. Jansson (1997). Antisense inhibition of photosystem I antenna protein Lhca4 in Arabidopsis thaliana. Plant Physiol. 115:1525-1531.

What is claimed is:

1. A process of increasing the level of phytosterol ester in plant seed beyond that of wild-type plant seed, said process comprising:
transgenically overexpressing an acyl-CoA sterol acyltransferase in a plant producing said plant seed.

2. The process of claim 1, wherein the level of phytosterol ester is increased in the plant seed by at least 10%.

3. The process of claim 1, wherein the level of phytosterol ester is increased in the plant seed by at least 25%.

4. The process of claim 1, wherein the level of phytosterol ester is increased in the plant seed by at least 30%.

5. The process of claim 1, wherein the phytosterol ester is cycloartenol.

6. The process of claim 1, wherein the plant is of a species selected from the group consisting of borage (Borago spp.), Canola, castor (Ricinus communis); cocoa bean (Theobroma cacao), corn (Zea mays), cotton (Gossypium spp.), Crambe spp., Cuphea spp., flax (Linum spp.), Lesquerella and Limnanthes spp., Linola, nasturtium (Tropaeolum spp.), Oeanothera spp., olive (Olea spp.), palm (Elaeis spp.), peanut (Arachis spp.), rapeseed, safflower (Carthamus spp.), soybean (Glycine and Soja spp.), sunflower (Helianthus spp.), tobacco (Nicotiana spp.), Vernonia spp., wheat (Triticum spp.), barley (Hordeum spp.), rice (Oryza spp.), oat (Avena spp.) sorghum (Sorghum spp.), rye (Secale spp.) or other members of the Gramineae.

7. The process of claim 1, further comprising: incorporating, for expression in the plant, a nucleic acid sequence selected from the group consisting of a nucleic acid sequence encoding a peptide having HMG-CoA reductase activity, a nucleic acid sequence encoding SMT1, a nucleic acid sequence encoding a peptide having mevalonate kinase activity, a nucleic acid sequence encoding a peptide that enhances early stages of phytosterol biosynthesis, a nucleic acid sequence encoding a peptide having sterol methyltransferase activity, a nucleic acid sequence encoding a peptide having squalene synthetase activity, a DNA to suppress expression of squalene epoxidase, a nucleic acid sequence encoding a C-14 sterol reductase peptide for the genetic manipulation of the plant sterol biosynthetic pathway, and any combination thereof.

8. A process of obtaining seeds, said process comprising:
(a) transforming a plant by:
i. transforming a plant cell with a recombinant DNA construct comprising a nucleic acid segment encoding acyl-CoA sterol acyltransferase and a promoter for driving the expression of said nucleic acid segment in said plant cell to form a transformed plant cell,
ii. regenerating the transformed plant cell into a transgenic plant, and
iii. selecting transgenic plants that have enhanced levels of phytosterol ester in the seeds compared wild type strains of the same plant;
(b) cultivating the transformed plant for one or more generations; and
(c) harvesting seeds from plants cultivated per (b).

9. The process of claim 8, further comprising:
further transforming the plant cell with a recombinant nucleic acid construct comprising a nucleic acid sequence selected from the group consisting of:
a nucleic acid sequence encoding a peptide having HMG-CoA reductase activity,
a nucleic acid sequence encoding SMT1,
a nucleic acid sequence encoding a peptide having mevalonate kinase activity,
a nucleic acid sequence encoding a peptide that enhances early stages of phytosterol biosynthesis,
a nucleic acid sequence encoding a peptide having sterol methyltransferase activity,
a nucleic acid sequence encoding a peptide having squalene synthetase activity,
a DNA to suppress expression of squalene epoxidase,
a nucleic acid sequence encoding a C-14 sterol reductase peptide for genetic manipulation of the plant cell's sterol biosynthetic pathway, and
any combination thereof, together with
a promoter for driving the expression of said nucleic acid segment in said plant cell.

10. A seed having enhanced levels of cycloartenol and produced by a plant having increased acyl-CoA sterol acyltransferase activity.

11. The seed of claim 10 wherein the seeds have a total level of sterol esters of at least 0.400% of dry weight.

12. A process for obtaining oil comprising enhanced levels of cycloartenol, said process comprising:
extracting oil from the seed of claim 10.

13. Oil produced by the process of claim 12.

14. A composition comprising the oil of claim 13, wherein the composition is selected from the group of a food product, a pharmaceutical composition, and a nutraceutical composition.

15. A. process of increasing sterol levels in seeds of plants and/or decrease cholesterol levels in plant tissue by increasing expression of acyl-CoA sterol acyltransferase in said plants.

16. The process of claim 15, further comprising:
increasing expression in the plant of a peptide having HMG-CoA reductase activity, SMT1, a peptide having mevalonate kinase activity, a peptide that enhances early stages of phytosterol biosynthesis in the plant, a peptide having sterol methyltransferase activity, a peptide having squalene synthetase activity, a DNA to suppress expression of squalene epoxidase, a C-14 sterol reductase peptide for the genetic manipulation of the plant sterol biosynthetic pathway, and any combination thereof.

17. Plant tissue having increased levels of cycloartenol, said plant tissue being derived from a plant having increased acyl-CoA sterol acyltransferase activity.

18. A process for modulating phytosterol synthesis in a plant, said process comprising:
modulating the expression of acyl-CoA sterol acyltransferase in the plant so as to modulate phytosterol synthesis therein.

19. A seed of the type having a mixture of phytosterols therein, the improvement comprising:
having cycloartenol as the most prominent phytosterol in said seed.

20. A process of obtaining seeds, said process comprising:
(a) transforming a plant by:
i. transforming a plant cell with a recombinant DNA construct comprising a nucleic acid segment encoding a peptide comprising SEQ ID NO:8 and having acyl-CoA sterol acyltransferase activity and a promoter for driving the expression of said nucleic acid segment in said plant cell to form a transformed plant cell,
ii. regenerating the transformed plant cell into a transgenic plant, and
iii. selecting transgenic plants that have enhanced levels of phytosterol ester in the seeds compared wild type strains of the same plant;
(b) cultivating the transformed plant for one or more generations; and
(c) harvesting seeds from plants cultivated per (b).
